(19) United States
(12) Patent Application Publication
(10) Pub. No.: US 2020/0330409 A1
(43) Pub. Date: Oct. 22, 2020

(54) INJECTABLE COMPOSITION COMPRISING HYALURONIDASE FOR REMOVING LOCAL FAT

(71) Applicant: Youna Son, Seoul (KR)

(72) Inventor: Youna Son, Seoul (KR)

(21) Appl. No.: 16/592,913

(22) Filed: Oct. 4, 2019

(30) Foreign Application Priority Data
Apr. 17, 2019 (KR) 10-2019-0044869

(51) Int. Cl. A61K 31/167; A61K 31/4402; A61K 31/137; A61K 31/375; A61K 31/205; A61K 33/00

(52) U.S. Cl. CPC A61K 31/167; A61K 31/4402; A61K 31/137; A61K 9/0019; A61K 31/205; A61K 33/00; A61K 31/375

(57) ABSTRACT

The present disclosure relates to an injectable composition comprising hyaluronidase for local fat reduction. More specifically, the present disclosure relates to an injectable composition comprising 300 IU to 600 IU of hyaluronidase and a local anesthetic, an antihistamine, a lipolysis stimulator, a neurotransmitter, a lipolytic agent, a treatment for allergies and acidosis, and a collagen production stimulator.

The present disclosure promotes the reduction of local fat tissues, thereby alleviating obesity and helping weight loss and maintenance of body shape while preventing side effects or skin imbalance by the even removal of fat. Further, at the same time, the present disclosure can achieve a skin lifting effect where skin resiliency is maintained by the stimulation of collagen production.

### CROSS-REFERENCE TO RELATED APPLICATIONS

The present application claims the benefit of priority from Korean Patent Application No. 10-2019-0044869 filed on Apr. 17, 2019, the entire disclosure of which is incorporated herein by reference.

### TECHNICAL FIELD

The present disclosure relates to an injectable composition for removing local fat. More specifically, the present disclosure relates to an injectable composition comprising hyaluronidase and one or more local anesthetics, antihistamines, neurotransmitters, lipolytic agents, lipolysis stimulators, pH adjusting agents, and collagen production stimulators.

The composition of the present disclosure helps to bum excess fat in the body, achieving the effect of aerobic exercise, and subsequent excretion of the metabolites leads to weight loss and improved body shape. The composition of the present disclosure also increases the basal metabolic rate, thereby enhancing the effects of exercise and diet, and stimulates the reduction of local fat tissue, thereby alleviating obesity and helping weight loss and the maintenance of body shape, while preventing side effects from the use of drugs or skin imbalance by the even removal of fat, and achieving a skin lifting effect where skin resilience is maintained by the stimulation of collagen production.

### BACKGROUND

With the recent increase in the obese population, obesity is gaining more attention, the number of obesity clinics is growing, and various social problems arising from obesity are becoming an issue.

Simply speaking, obesity is defined as the over-accumulation of body fat and causes adult diseases such as hypertension, diabetes, and hyperlipidemia, constituting a major part of the metabolic syndrome.

In particular, women tend to desire a slimmer and more balanced body line, even when they are not obese.

In order to remove local fats such as abdominal fat, etc. accumulated from obesity, a surgical procedure called liposuction is widely used.

However, such surgical procedure may cause severe side effects, such as wounds at the operated sites, edema, paralysis and burning sensation, risk of infection, other injuries to skin or nerves, or perforation of vital organs.

In addition, liposuction requires a substantial amount of recovery time, and there are always serious anesthesia-related risks since surgical procedures like liposuction require local anesthesia or general anesthesia, depending on the case.

For the reasons explained above, various lipolysis stimulators which break down local fats have been used recently to improve apparent obesity resulting from the accumulation of body fats.

Lipolysis refers to the process in which triglycerides are hydrolyzed into free fatty acids (FFA) and glycerol by the action of hormone sensitive lipase (HSL) accumulated in adipocytes.

The lipolytic agent hyaluronidase, which is the collective name for enzymes catalyzing the degradation of hyaluronic acid, was first known as a dispersion component by Duran-Reynals, but, as it was later observed to exhibit strong activity on hyaluronic acid (HA), it came to be called hyaluronidase (HAase).

Hyaluronidase is known to hydrolyze the linkage between N-acetyl-D-glucosamine and D-glucuronic acid present in hyaluronic acid (HA), chondroitin, and chondroitin sulfates.

Hyaluronidase can accumulate in the dermis and depolymerize long chain mucopolysaccharides which are causing the retention of associated water and the delay of organic fluid diffusion removing metabolic waste products by microvascular constriction.

Depolymerization of mucopolysaccharides cleaves their long chains into short chains, resulting in the loss of associated water and waste products as well as recovery of venous and lymphatic circulation, thereby achieving the dissipation of localized edema. Utilizing such connective tissue lysing action, hyaluronidase is used in methods of local fat removal by injecting it subcutaneously to loosen abdominal connective tissue, thereby reducing edema in the tissue and consequently helping blood and lymph circulations so that the degradation and metabolism of abdominal adipose cells are facilitated.

Among the above methods of local fat removal by lipolysis using hyaluronidase, LLD (Lipolytic Lymph Drainage) treatment is widely used (KR 10-2009-0111916A).

LLD treatment destroys some adipose cells and causes the accumulated fat-related substances to rapidly move to the lymph vessel to be oxidized and excreted, consequently reshaping the overall body line.

However, in the above document, a high dose (1500 IU) of hyaluronidase was used, which makes it difficult to remove fat evenly over a wide area, resulting in dimpling, sagging and wrinkles in the skin. Also, the side effects of the drug make continuous treatment difficult.

In addition, KR 10-2013-0003394A discloses an injectable composition preventing pain and allergies by mixing 1 cc each of hyaluronidase, lidocaine which is a local anesthetic, and pheniramine which is an antihistamine. However, this composition also uses hyaluronidase at a high dose, and injection of the composition is performed on 4 to 6 points with a 5-cm interval between the injection points, thus still having the problem of not being able to avoid side effects such as dimpling, sagging, or wrinkling.

In an attempt to solve the problems of the above patent literatures, the inventor of the present disclosure developed an injectable composition for local fat removal comprising a low dose of hyaluronidase along with an antihistamine, a local anesthetic, a lipolysis stimulator, and a collagen production stimulator, which was registered as KR 10-1706549.

A microneedle device for lipolysis of abdominal fat, which comprises components similar to the inventor's Korean patent mentioned above was registered as KR 10-1806074.

In addition, an anti-obesity composition comprising choline alfoscerate is disclosed in KR 10-2017-0094667A, and a composition for lipolysis injection comprising alfocholine and alpha-lipoic acid, L-carnitine, choline alfoscerate, vitamin B, etc., is disclosed in KR 10-1865914 and KR 10-1882400.

Although the lipolysis injectable compositions disclosed in the above patent literature have some efficacy in removing fat, their effects still fall short of satisfactory.

### SUMMARY OF THE INVENTION

It is an object of the present disclosure to provide an injectable composition for local fat removal which can increase the basal metabolic rate, thereby enhancing the effects of exercise and diet, and minimize side effects such as dimpling, sagging, or wrinkling by removing fat evenly over a wide area through the burning of excess fat, thus minimizing the side effects of injectable compositions used in LLD treatments to resolve the above-mentioned problems.

Further, it is an object of the present disclosure to provide an optimal injectable composition which can stimulate collagen production so that skin resiliency is maintained in the local area of the fat removal.

As an improvement of the inventor's earlier patent KR 10-1706549, the present disclosure has been completed, after a long period of clinical trials to enhance lipolysis and minimize the side effects of the hyaluronidase-containing injectable composition for fat removal disclosed in KR 10-1706549, by adding to the lipolysis action of said injectable composition components that help burn excess fat in the body, achieving the effect of aerobic exercise as well as weight loss and improved body shape through subsequent excretion of the metabolites, and also increase the basal metabolic rate, thereby enhancing the effects of exercise and diet.

That is, the present disclosure is characterized by further incorporating epinephrine, a neurotransmitter that activates lipase, which hydrolyzes triglycerides into free fatty acids and glycerol, while reducing insulin which inhibits the lipase, choline alfoscerate, which is a lipolytic agent that reduces adipose cells by lipolysis, and a bicarbonate, a pH adjusting agent for tissue protection by pH regulation in lipid metabolism and treatment of acidosis and allergies, into the injectable composition for fat removal according to the above-mentioned earlier patent.

### DETAILED DESCRIPTION

The components of the present disclosure will now be explained in detail by references to the disclosures in the present inventor's earlier patent KR 10-1706549.

As described in the above patent, the present disclosure uses hyaluronidase in an amount between 300 IU to 600 IU, its optimal dose to facilitate lipolysis based on experimental results showing that, at 300 IU or below, while it was possible to dissolve fillers, sufficient lipolysis did not occur, and at above 600 IU, while excellent effects were obtained in terms of fat removal, side effects were observed which was the problem with conventional injectable compositions where the skin became bumpy due to dimpling resulting from excessive lipolysis. It was ascertained from clinical trials that at the hyaluronidase unit dose of 300 IU to 600 IU, it was possible to ensure adequate fat removing effect while minimizing side effects. It was also shown in clinical trials that at the above dose range, if the injectable composition is administered in a volume of 1 cc containing 300 IU of hyaluronidase, the diameter of the diffusion range is about 1 cm. Thus, with the hyaluronidase dose set at 300 IU to 600 IU, by injecting the composition at a volume of 0.5 to 2 cc depending on the hyaluronidase dose and with an interval of 0.5 to 1.5 cm between injection points, injection at narrow intervals is possible, and consequently, lipolysis occurs evenly. Furthermore, a lifting effect occurs concomitantly where skin resilience is improved by the promotion of collagen production through the stimulation of skin by the injection needle.

In addition, the present disclosure provides an injectable composition which further comprises a local anesthetic to alleviate the pain from the injection, an antihistamine to prevent allergies, a lipolysis stimulator to promote lipolysis, and a collagen production stimulator.

As the local anesthetic, lidocaine can be used. Local anesthetics should be used in minimal amounts, as they may exhibit toxicity when used in excessive amounts. Clinical trials showed that the most preferable dose is 0.80 to 4.00 mg per 1 cc of injectable composition. At a dose below the above-mentioned value, anesthetic effect is unsatisfactory, and at a dose above the above-mentioned value, patients experience inconveniences in daily life as it takes more than 2 hours to fully recover sensation after an injection, and the probability of side effects due to lidocaine increases.

As the antihistamine of the present disclosure, first generation antihistamines were selected since the antihistamine should exhibit an immediate effect during the injection. Among these, those that can be administered subcutaneously include piprinhydrinate (=diphenylpyridine) and chlorpheniramine, with chlorpheniramine found to be more preferable, having less side effects and a shorter duration of action.

The amount of the antihistamine used is 0.10 to 0.20 mg per 1 cc of injectable composition. At a dose below the above-mentioned value, antihistamine effect is unsatisfactory, and above it, side effects due to the antihistamine may occur.

As the lipolysis stimulator, L-carnitine can be used. L-carnitine is a type of vitamin and a nutrient essential for energy production and fat oxidation. In adults, it is synthesized in the liver and kidneys and can be taken from animal source food or dietary supplements.

L-camitine is an essential substance for the transportation of fatty acids across the mitochondrial membrane to stimulate β-oxidation, and carnitine palmitoyltransferases I and II and acylcarnitine transferase are involved in the action of L-carnitine.

Recent research shows that carnitine palmitoyltransferase I is an important rate-limiting enzyme in fat oxidation and plays a key role in the utilization of substrates for energy production.

L-camitine is also a modulator of glucocorticoid receptor functions and plays a role similar to a glucocorticoid, stimulating lipolysis in the adipose tissue.

As the lipolysis stimulator, L-carnitine can be used. Although L-camitine is produced in the body, the amount is not sufficient, and consequently, it needs to be supplied externally. Since L-carnitine is mostly present in animal proteins, a strict vegetarian diet may lead to carnitine deficiency.

Thus, while L-camitine is essential for facilitating the degradation of body fat and increasing metabolism to alleviate obesity, it can be easily deficient if not supplied externally.

In particular, when one cuts back on the intake of animal proteins during a period of dieting, L-carnitine almost inevitably becomes deficient, with the rate of fat burning reduced as well as the metabolic rate. Therefore, in order to enhance the effect of dieting and alleviate metabolic diseases, the addition of L-carnitine is thought to be necessary.

However, L-carnitine can cause side effects such as nausea, vomiting, upset stomach, diarrhea, and body odor. It can also cause seizures and increase the frequency and intensity of seizures in patients with a history of seizures. As a result of repeated studies to find doses that promote the metabolism of fat while minimizing the above side effects, it has been found that L-carnitine can be administered at an amount of 0.1 to 40 mg per 1 cc of injectable composition.

In addition, using L-carnitine together with a lipolytic agent such as phosphatidylcholine, aminophylline, caffeine, or the like can further improve the lipolytic effect.

Furthermore, in order to prevent skin sagging, which is the biggest problem in lipolysis and dieting, and make a resilient and beautiful body line, non-toxic vitamin C was selected as a collagen production stimulator.

Vitamin C has the effect of increasing the resiliency of skin by collagen production and is a natural antihistamine. Thus, it was necessary to find the minimal effective dose to prevent side effects associated with administration of excessive amounts. Clinical trials showed that 0.1 to 20 mg of vitamin C per 1 cc of injectable composition is most preferable.

Epinephrine, also called adrenaline, is a substance produced in the adrenal medulla which acts both as a hormone and a neurotransmitter. It also acts to prevent bleeding through its pharmacological activity of causing constriction of the peripheral blood vessels.

Excess nutrients absorbed into the body are stored in the form of a triglyceride (TG).

Having the form where three fatty acids are bound to one glycerol, TG is hydrolyzed by a lipid hydrolyzing enzyme, lipase, into one glycerol and three fatty acids, which is called lipolysis.

Various hormones are involved in lipolysis, including low insulin, increased glucagon and epinephrine secretion, among others (growth hormones, glucagon, thyroid hormone, etc.).

Binding of a catabolic hormone like epinephrine to a receptor on the cell membrane activates the enzyme adenylate cyclase, which converts adenosine triphosphate (ATP) to adenosine monophosphate (cAMP).

Increased cAMP in the cell activates protein kinase A (PKA), and PKA activates the lipid hydrolyzing enzyme lipase. The lipase whose activation by PKA is attributable to hormones is thus called hormone-sensitive lipase (HSL).

TG is mobilized by HSL, releasing one glycerol and FFA into the blood stream.

Thus, the above-mentioned hormones are involved in the metabolic pathways through which fat is used as the energy source, during exercise or ordinary activities.

Most of the glycerol transported to the liver via the blood stream is converted by glycerol kinase to glycerol-3-phosphate (G-3-P), which is then converted to dihydroxyacetone phosphate (DHAP), which then becomes a part of glucose (Gluconeogenesis).

Since muscles have low glycerol kinase activity, most of the glycerol is absorbed at the liver. Some glycerol, however, is converted to pyruvic acid via DHAP and then used as an energy source.

DHAP can also be converted to G-3-P via catalysis by NAD-linked glycerol-3-phosphate dehydrogenase.

Epinephrine, an additional component in the composition of the present disclosure, activates the lipid hydrolyzing enzyme lipase and decreases insulin, which suppresses the activation of lipase.

In particular, epinephrine bound to α-adrenergic receptor inhibits insulin secretion, thereby stimulating the breakdown of glycogen, and stimulates glycolysis in muscle. Binding to β-adrenergic receptor stimulates glucagon secretion and increases adrenocorticotropic hormone (ACTH) secretion by the pituitary gland.

In addition, epinephrine triggers increased lipolysis by the adipose tissue. These actions lead to increased glucose and fatty acids in the blood, providing them for energy production in cells throughout the body.

Thus, epinephrine can be considered essential for effective breakdown of body fat and improving obesity since its role as neurotransmitter in sympathetic responses and as a hormone leads to increased basal metabolic rate, thereby giving an effect of aerobic exercise and diet. Based on this, epinephrine is selected as a component of the injectable composition according to the present disclosure.

Epinephrine also prevents bleeding from subcutaneous injection or excessive lipolysis through its peripheral vasoconstriction effect, thereby minimizing side effects including bruising.

The amount of epinephrine used is 0.01 to 0.08% by weight based on the total weight of the injectable composition. At an amount below the above-mentioned value, insulin suppression and lipase activation is unsatisfactory, resulting in a lower lipolysis effect, and above it, side effects may occur including bruising due to excessive lipolysis and increased blood pressure.

Choline alfoscerate is a natural choline compound found in the brain that is a precursor of acetylcholine. It is known to have potential for the treatment of Alzheimer's disease and other dementias.

Choline alfoscerate rapidly delivers choline to the brain across the blood-brain barrier (BBB) and is a biosynthetic precursor of acetylcholine. It is a drug proven safe and used in most countries. Choline is a structural element in cell membranes and plays a role in neurotransmission as the precursor for acetylcholine. It is known as a major source of methyl groups.

Thus, choline alfoscerate facilitates choline supply for the synthesis of acetylcholine, thereby improving neurotransmission to recover normal nerve functions. At the same time, the supply of glycerophosphate acts on the phospholipids in nerve membranes to recover nerve cell membranes and improve nerve functions.

Choline alfoscerate is a choline precursor like some other drugs used for fat removal, such as phosphatidylcholine(PPC) or deoxycholine. Having a lipodissolve effect, it is effective in decreasing adipose cells.

Phosphatidylcholine given as PPC injection is accompanied by excess edema lasting over one week, and reported sequelae include pigmentation due to bruising from tissue injury occurring in the process of fat removal. Further, severe problems such as skin necrosis may result in some cases. As a result, it cannot be used anymore as its approval was revoked.

Choline alfoscerate is suitable as an injection for the face area because it has less of the side effects mentioned above. Also, as a choline precursor, it may have potential for lipolysis effect. Having ascertained from the experimental studies that choline alfoscerate is free from serious side effects and has a satisfactory effect in decreasing adipose cells, the present inventor has included it in the injectable composition of the present disclosure.

The amount of choline alfoscerate used is 0.01 to 4.0% by weight based on the total weight of the injectable composition. At an amount below the above-mentioned value, decrease in adipose cells is unsatisfactory, and above it, side effects may occur.

Sodium bicarbonate (Bivon; NaHCO₃) is a key component in the pH buffering system (acid-base homeostasis) in the body and plays an important biochemical role in the physiological pH buffering system.

Sodium bicarbonate is used for pH adjustment upon lipolysis in the body and as a treatment for acidosis and acute hives. In the present disclosure, it is added in an amount of 0.05 to 0.3% by weight pH adjustment effect after lipolysis by the injectable composition, and to prevent side effects such as allergies.

The injectable composition of the present invention comprises, based on a volume of 1 cc, 300 IU to 600 IU of hyaluronidase, 0.80 to 4.00 mg of a local anesthetic, 0.10 to 0.20 mg of an antihistamine, 0.1 to 40 mg of a lipolysis stimulator, 0.1 to 20 mg of a collagen production stimulator, 0.20 to 0.50 mg of the neurotransmitter epinephrine, 0.1 to 30 mg of the lipolytic agent choline alfoscerate, and 0.60 to 4.00 mg of sodium bicarbonate as allergies and acidosis treatment, with the remainder being a saline solution.

The above amounts were determined in consideration of the fact that the daily dose is 15,000 IU for hyaluronidase, 200 mg for local anesthetics, 10 mg for antihistamine, 2000 mg for lipolysis stimulator, 1000 mg for collagen production stimulator, 15 mg for epinephrine, 1500 mg for choline alfoscerate, and 300 mg for sodium bicarbonate. Amounts exceeding the above ranges may cause side effects.

According to the present disclosure, by adding the neurotransmitter epinephrine to a prior art injectable composition for fat removal, lipase catalyzing the lipolysis is activated and the production of insulin, which suppresses the lipase, is suppressed. As a result, the injectable composition of the present disclosure can persistently stimulate lipolysis and increase blood glucose and fatty acids, providing them for energy production in cells throughout the body to give the effect of aerobic exercise, and promoting energy metabolism and subsequent excretion of fat to give an effect of spontaneous weight loss. In addition, peripheral vasoconstriction effect prevents subcutaneous bleeding, thereby preventing side effects such as bruising. Addition of choline alfoscerate having fewer side effects gives a steady lipolysis effect, and sodium bicarbonate, playing an important biochemical role in the physiological pH buffering system in the body, can minimize side effects of the injectable composition by its pH adjustment function after lipolysis, as well as its effect in the treatment of acidosis and acute hives.

The injectable composition of the present disclosure may further comprise, without limitation, placenta components that are excellent in skin tissue regeneration and anti-aging or pentoxifylline which improves blood circulation, within dose ranges allowable for injectable compositions.

The injectable composition of the present disclosure as described above is useful in that it has an excellent effect in local fat removal, as it allows narrow interval injection, has a steady lipolysis effect based on the effect of aerobic exercise and dieting from increased basal metabolic rate, and minimizes side effects to enable continued treatment, prevention of dimpling by even fat removal, and maintaining skin resilience by collagen production.

The injectable composition of the present disclosure helps to burn excess fat in the body, achieving the effect of aerobic exercise, and subsequent excretion of the metabolites leads to spontaneous weight loss and improved body shape. The composition of the present disclosure also increases the basal metabolic rate, thereby enhancing the effects of exercise and dieting, minimizes side effects such as dimpling or sagging and wrinkling by removing fat evenly over a wide area through the burning of excess fat, allows continued obesity treatment by minimizing side effects, and enables slim and smooth skin regeneration without dimpling by even fat removal.

By collagen regeneration, the present disclosure also prevents skin sagging or wrinkling due to fat removal, thereby achieving skin lifting effect which allows skin resilience to be maintained.

The present disclosure will now be described in detail with references to specific working examples.

Hyaluronidase is a water-soluble enzyme secreted from mammalian vas deferens or testes, characterized by its actions to remove barriers between tissues by hydrolyzing the glucosaminic bonds between hyaluronic acid, a major intercellular substance, and connective tissues to dissolve the bonds and reduce fibroplasia in tissues. Hyaluronidase is also known to relieve swelling and edema in tissues.

Using the characteristics of hyaluronidase that hydrolyzes and dissolves substances within skin tissues, LLD (Lipolytic Lymph Drainage) treatment is widely used in which hyaluronidase is directly injected subcutaneously to degrade substances including fibers in subcutaneous tissues, thereby facilitating lymphatic circulation to help circulation of adipose tissues to reduce adipose tissues and relieve obesity.

However, the above treatment, though helpful in reducing adipose tissues, has been associated with various side effects.

That is, using high doses of hyaluronidase to remove adipose tissue causes allergic symptoms such as redness or itching, and excessive lysis of adipose tissues leads to bruising due to subcutaneous bleeding or dimpling, resulting in frequent discontinuation of the LLD treatment to remedy these side effects and difficulty of continued treatment due to patients' repulsion arising from pain.

Furthermore, using high doses of hyaluronidase makes it difficult to narrow the intervals between injection points (about 5-cm intervals). As a result, fat removal is concentrated only around injection points to cause indentations or sagging and wrinkling in the skin, and these side effects lead to imbalance. Significant reduction in skin resiliency also results in the dissatisfaction of patients.

Having strived to address the above problem, the present inventor discovered that the minimum amount of hyaluronidase exhibiting efficacy is 300 IU, and that above 600 IU, adipose cells are excessively hydrolyzed, giving rise to concerns about dimpling or subcutaneous bleeding. Thus, by administering an injectable composition of the present disclosure having the above compositional ranges, the side effects from drug use are minimized while fat removal over a wide area can be carried out using minimal volumes.

It was shown in clinical trials that at a hyaluronidase dose of 300 IU, the diameter of the diffusion range for the injectable composition is about 1 cm. Thus, by injecting the composition at narrow intervals between injection points of 0.5 to 2 cm, even fat removal as well as collagen production by skin stimulation can be facilitated.

Also, by comprising a local anesthetic to alleviate pain from the injection, an antihistamine to prevent allergic side effects, a lipolytic agent, a lipolysis stimulator to rapidly transport fatty acids from the degraded fat into mitochondria to further degrade them, a neurotransmitter whose function in sympathetic responses and as hormone facilitates effective breakdown of body fat, and which prevents subcutaneous bleeding, an agent for pH adjustment and treatment of allergies and acidosis, and a collagen production stimulator, the injectable composition of the present disclosure also prevents allergic symptoms like redness or itching as well as pain and bruising, accelerates fat removal, and excels in maintaining skin resiliency by collagen production.

As the above local anesthetic, drugs commonly used in injectable solutions can be used. The present disclosure uses lidocaine in an amount of 0.08 to 0.4% by weight based on the total weight of the composition.

When the amount of the local anesthetic is below 0.08% by weight, the analgesic effect is unsatisfactory, and when the amount is above 0.4% by weight, the duration of the anesthesia is prolonged and is uneconomical.

As for the other ingredients, the antihistamine pheniramine is contained in an amount of 0.01 to 0.02% by weight, the lipolysis stimulator L-carnitine in an amount of 0.01 to 4.0% by weight, the neurotransmitter epinephrine in an amount of 0.01 to 0.08% by weight, the lipolytic agent choline alfoscerate in an amount of 0.01 to 4.0% by weight, sodium bicarbonate for pH adjustment and as allergies and acidosis treatment in an amount of 0.05 to 0.3% by weight, and the collagen production stimulator vitamin C in an amount of 0.1 to 2.0% by weight.

The injectable composition of the present disclosure may also comprise one or more substances selected from a tonicity agent, a nonionic surfactant, a stabilizer, a preservative, etc., that are commonly used in injectable solutions, within dose ranges allowable for injectable compositions. The composition of the present disclosure may further comprise placenta components or pentoxifylline which improves blood circulation, in amounts of 0.01 to 4.0% by weight.

The amounts of the above ingredients were determined within their respective maximum daily doses in preparation for a case where 300 IU of hyaluronidase is administered at a daily dose of 15,000 IU.

Most preferably, the injectable composition of the present disclosure is injected to give 300 IU to 600 IU of hyaluronidase per injection, at a unit volume of 0.5 to 2 cc with an interval between injection points of 0.5 to 1.5 cm. The daily dose of hyaluronidase is preferably 15,000 IU or less.

The unit dose and injection points described above can be suitably adjusted according to the unit of hyaluronidase given in one injection.

The injectable composition of the present disclosure can be injected 50 times based on a volume of 1 cc containing 300 IU of hyaluronidase, and the site of injection can be any body area where fat has accumulated, including the abdomen, buttocks, thighs, calves, chin, forehead, arms, and the like.

### EXAMPLES

Preparation of injectable compositions

Ingredients and composition ratios of the injectable compositions are described in Table 1, with a composition containing hyaluronidase in an amount of less than 300 IU designated as Comparative Example 1, a composition containing hyaluronidase in an amount of greater than 600 IU designated as Comparative Example 2, a composition containing hyaluronidase in an amount of 300 IU designated as Comparative Example 3, and a composition according to the present disclosure designated as the Example.

TABLE 1

| | Comp. | Comp. | Comp. | |
|---|---|---|---|---|
| | Example | Example | Example | |
| Ingredients | 1 | 2 | 3 | Example |
| Hyaluronidase | 25,000 IU | 65,000 IU | 30,000 IU | 30,000 IU |
| Lidocaine | 21.0 mg | 21.0 mg | 21.0 mg | 21.0 mg |
| Pheniramine | 1.2 mg | 1.2 mg | 1.2 mg | 1.2 mg |
| L-Carnitine | 66.0 mg | 66.0 mg | 66.0 mg | 66.0 mg |
| Vitamin C | 60.0 mg | 60.0 mg | 60.0 mg | 60.0 mg |
| Epinephrine | | | | 5.0 mg |
| Choline | | | | 40.0 mg |
| alfoscerate | | | | |
| Sodium | | | | 20.0 mg |
| bicarbonate | | | | |
| Saline solution | q.s. | q.s. | q.s. | q.s. |
| Total Amount (cc) | 100 | 100 | 100 | 100 |

For the hyaluronidase, Liporase of Daehan New Pharm Co., Ltd (1500 IU/vial, including 13.3 mg lactose hydrate) was used, to which lidocaine (Hanmi Pharm. Co., Ltd.), pheniramine (Yuhan Co.), L-carnitine (Dream Pharma), epinephrine (Daihan Pharm.), Alfocholine (Daewon Pharmaceutical), sodium bicarbonate (Daewon Pharmaceutical), and vitamin-C (Daewoo Pharm Co., Ltd.) were added in the amounts listed in the above table, and the mixture was dissolved in saline solution to prepare 100 cc of a solution composition for injection.

### Experimental Example 1

Lipolytic effect of the injectable composition of the present disclosure

Changes in waist circumference after the administration of the injectable composition of the Example and those of Comparative Examples 1 to 3, as well as after endomology treatment for edema were measured and presented in Table 2.

The subjects of treatment were a total of 40 patients (20 male, 20 female) having abdominal obesity with a BMI of 25 or greater, selected from adult outpatients of age 20 or above.

In the abdominal area between the ribs and pubic bones, 1 cc each of an injectable composition was injected at 50 points, arranged in a manner that they are apart 1 cm each starting from the navel. The above injection was repeated weekly for four weeks, with the points of injection moved each time. Decreases in waist circumference were measured 1 week after completion of the treatment, and then again 1 month after completion of the treatment. The average values of the measurements were recorded.

Endomology treatment, designed for edema, was carried out for four weeks, one 30-minute session per week, with the decrease in waist circumference measured after one week and one month, and the results were recorded.

For statistical analysis, the statistics package SPSS was used to obtain the distribution of measurements and the paired t-test was used to assess the effects of test injections on the decrease in waist circumference. The analysis used a 95% confidence interval, adjusted for weight changes.

TABLE 2

| | | | Diff- | | Diff- |
|---|---|---|---|---|---|
| | | 1 week | erence | 1 month | erence |
| | | after 4 | after 1 | after 4 | after 1 |
| | Baseline | sessions | week | sessions | month |
| Example | 90.7 ± | 78.1 ± | −12.6 ± | 78.3 ± | −12.4 ± |
| | 1.2 | 0.3 | 0.2 | 0.2 | 0.3 |
| Comp. | 91.0 ± | 89.4 ± | −1.6 ± | 89.8 ± | −1.2 ± |
| Example 1 | 1.0 | 0.4 | 0.6 | 1.3 | 0.3 |
| Comp. | 90.7 ± | — | — | — | — |
| Example 2 | 1.3 | | | | |
| Comp. | 90.8 ± | 81.4 ± | −9.4 ± | 81.3 ± | −9.5 ± |
| Example 3 | 1.7 | 0.5 | 0.2 | 0.3 | 0.4 |
| End- | 92.0 ± | 89.8 ± | −2.2 ± | 92.2 ± | +0.2 ± |
| omology | 2.0 | 1.4 | 0.8 | 1.2 | 1.5 |

[symbol]

In the Example, body fat analysis using the body fat analyzer Inbody showed an average fat loss of 2.5 to 3.2 kg.

According to Table 2, in Comparative Example 1 where 250 IU of hyaluronidase was administered, the difference was −1.6±0.6 cm at one week after the treatment and −1.2±0.3 cm at one month after the treatment. In Comparative Example 2 where 650 IU of hyaluronidase was administered, the treatment was discontinued after the first session due to dimpling and bruising. In Comparative Example 3, which is according to the earlier patent of the present inventor, a difference of −9.4±0.2 cm in waist circumference was seen at one week after the four sessions. In the Example according to the present disclosure, however, a difference of −12.6±0.2 cm in waist circumference was seen at one week after the four sessions, and the difference was still −12.4±0.3 cm after one month, indicating that there was no yo-yo effect.

As can be seen from the above table, the injectable composition of the present disclosure shows about 30% or more stronger effect in reducing the waist circumference compared to Comparative Example 3, which was prepared according to the earlier patent.

In the case of endomology, a decrease in waist circumference from the removal of edema was not significant and disappeared after one month, clearly indicating that a decrease in waist circumference achieved by the present disclosure is not only the result of edema removal, but also from lipolysis.

As can be seen from the above results, the injectable composition of the present disclosure has been demonstrated to have an excellent effect in local fat removal, with remarkably reduced side effects and no yo-yo effect.

### Experimental Example 2

Assessment of side effects of the injectable composition of the present disclosure

The injectable compositions of the Example and those of Comparative Examples 1 to 3 were administered as in Experimental Example 1, and the side effects observed in each patient in clinical trials are indicated in Table 3.

TABLE 3

| | | | | Pain above | | | | Skin |
|---|---|---|---|---|---|---|---|---|
| | Redness | Swelling | Itching | VAS 3 | Bruising | Dimpling | Sagging | resilience |
| Comp. | 1 | 1 | 0 | 0 | 0 | 0 | 0 | 3 |
| Example 1 | | | | | | | | |
| Comp. | 4 | 3 | 4 | 2 | 10 | 15 | 0 | 2 |
| Example 2 | | | | | | | | |
| Comp. | 1 | 0 | 0 | 0 | 0 | 0 | 0 | 5 |
| Example 3 | | | | | | | | |
| Example | 0 | 0 | 0 | 0 | 0 | 0 | 0 | 5 |

* Skin resilience was determined by tactile assessment using a 5-point scale: 1: Very poor, 2: Poor, 3: Fair, 4: Good, 5: Very Good.

As shown in Table 3, the injectable composition of the Example according to the present disclosure was shown to have remarkably reduced allergic redness, swelling, itching and pain compared with Comparative Examples 1 to 3.

These effects are thought to result from the use of a local anesthetic, an antihistamine, epinephrine, alfocholine and sodium bicarbonate as well as the low dose hyaluronidase.

Taken all together, the above effects are thought to result from the minimized unit dose of hyaluronidase, which allows for the administration of the injectable composition over a wide area leading to even removal of fat, and the stimulation of fat oxidation and steady lipolysis and reduction of side effects due to the use of the neurotransmitter epinephrine, the lipolytic agent choline alfoscerate, and the allergies and acidosis treatment sodium bicarbonate, as well as the enhanced skin resiliency due to the collagen production stimulator.

Hyaluronidase is an effective substance for local fat reduction that has no particular side effects. It is known to have an efficacy nearly comparable to other conventional drugs and shows few adverse interactions with other drugs. Thus, it is expected that hyaluronidase may maximize the therapeutic effect on obesity through combined administration with other drugs.

It is to be understood that the above detailed description and working examples are provided by way of illustration only, and are not to be taken as limitations.

Accordingly, a person of ordinary skill in the art would readily understand that various modifications and equivalents may be made without departing from the scope of the present disclosure.

Thus, the scope of protection for the present disclosure will be determined by the appended claims and equivalents thereof.

What is claimed is:

1. An injectable composition for local fat removal comprising 300 IU to 600 IU of hyaluronidase and, based on the weight of the total composition, 0.08 to 0.4% by weight of lidocaine as local anesthetic, 0.01 to 0.02% by weight of pheniramine as antihistamine, 0.01 to 4.0% by weight of L-carnitine as lipolysis stimulator, 0.01 to 0.08% by weight of the neurotransmitter epinephrine, 0.01 to 4.0% by weight of the lipolytic agent choline alfoscerate, 0.05 to 0.3% by weight of sodium bicarbonate for pH adjustment and as allergies and acidosis treatment, and 0.1 to 2.0% by weight of vitamin C as collagen production stimulator, with the remainder being saline solution, wherein the composition is characterized by being injected at a unit volume of 0.5 to 2 cc with an interval between injection points of 0.5 to 1.5 cm.
